(19) United States
(12) Patent Application Publication
(10) Pub. No.: US 2025/0114191 A1
(43) Pub. Date: Apr. 10, 2025

(54) TRANSCATHETER HEART PROSTHESIS

(71) Applicant: MEDTRONIC, INC., Minneapolis, MN (US)

(72) Inventors: Daniel Margolis, Rohnert Park, CA (US); Justin Goshgarian, Santa Rosa, CA (US); Curtis Goreham-Voss, Maple Grove, MN (US)

(21) Appl. No.: 18/729,930

(22) PCT Filed: Jan. 26, 2023
(86) PCT No.: PCT/US2023/061375

§ 371 Date: Jul. 18, 2024

Related U.S. Application Data
(60) Provisional application No. 63/481,395, filed on Jan. 25, 2023.
(60) Provisional application No. 63/303,675, filed on Jan. 27, 2022.

(51) Int. Cl. A61F 2/24

(52) U.S. Cl. CPC A61F 2/2418; A61F 2250/0037

(57) ABSTRACT

A transcatheter valve prosthesis includes a frame and a prosthetic valve coupled to the frame. In a radially compressed configuration, the frame is a single layer tube. In a radially expanded configuration, the frame comprises an outer structure and an inner structure, wherein the prosthetic valve attached to the inner structure, and wherein the outer structure surrounds the inner structure and is spaced from the inner structure by a gap.

### FIELD

The present technology is generally related to transcatheter heart valve prostheses, and, in particular, is directed towards transcatheter valve prostheses having a frame including an inner structure and an outer structure in a radially expanded configuration made from a singular or unitary tube such that in a radially compressed configuration the frame is a single-layer tube

### BACKGROUND

The human heart is a four chambered, muscular organ that provides blood circulation through the body during a cardiac cycle. Within the heart there are four valves that control blood flow through the heart's chambers: the mitral valve, the tricuspid valve, the aortic valve, and the pulmonary valve. To ensure blood flows in only one direction, atrioventricular valves (the tricuspid and mitral valves) are present between the junction of the atrium and the ventricles, and semi-lunar valves (pulmonary and aortic valves) govern the exits of the ventricles leading to the lungs and the rest of the body. Each of these valves contain native leaflets that open and close in response to changes in blood pressure as the heart contracts and relaxes. When a valve does not open or close properly, either due to defect or damage, diseases such as stenosis and valvular insufficiency or regurgitation can occur leading to serious physiological consequences.

Transcatheter heart valve prostheses have been developed for repair and replacement of diseased or damage heart valves. The heart valve prosthesis can be radially compressed or reduced in diameter for delivery to a treatment site via a delivery catheter and can be deployed at the treatment site of the disease heart valve. Once the heart valve prosthesis is positioned at the treatment site, for instance, within a mitral valve, the heart valve prosthesis can be radially expanded to hold the heart valve prosthesis in place.

Heart valve prostheses generally include a stent or frame and a prosthetic valve attached to the frame. In some heart valve prostheses, the frame includes an inner stent and an outer stent attached to each other, with the prosthetic valve attached to the inner frame and the outer frame engaging tissue at the treatment site. Such heart valve prostheses, referred to herein as “dual frame” or “dual stent” designs or prostheses, may be advantageous in that the outer stent may conform to the native tissue and absorb forces imparted on the heart valve prosthesis by the native tissue with minimal or no effect on the inner stent carrying the prosthetic valve. However, with such dual stent prostheses, when the heart valve prosthesis is in the radially compressed configuration for delivery, the inner and outer stents remain layered or stacked or coaxial one inside the other. Smaller packing or delivery profiles are desirable for delivering transcatheter heart valve prostheses to the treatment site. Such “dual stent” prostheses may be limited in further reducing the outer diameter thereof in the radially compressed configuration due to the layered inner and outer stents in the radially compressed configuration. Therefore, a heart valve prosthesis with the advantages of a dual stent prosthesis in the radially expanded configuration with a smaller packing profile is desirable.

### SUMMARY

According to a first aspect hereof, a transcatheter valve prosthesis includes a frame having a central longitudinal axis. The frame includes an inflow portion, an outflow portion, and a plurality of commissure bars extending from the outflow portion towards the inflow portion. A prosthetic valve is operatively connected to the plurality of commissure bars. In a radially compressed configuration, each of the plurality of commissure bars is disposed within a respective opening of a plurality of openings in the frame, and the inflow portion, the outflow portion, and the plurality of commissure bars are spaced a first distance from a central longitudinal axis of the frame. In a radially expanded configuration, the inflow portion is spaced a second distance from the central longitudinal axis greater than the first distance, and the plurality of commissure bars are spaced a third distance from the central longitudinal axis of the frame less than the second distance.

In a second aspect, in the transcatheter valve prosthesis according to first aspect, the plurality of commissure bars extends substantially parallel to the central longitudinal axis.

In a third aspect, in the transcatheter heart valve prosthesis according to the first aspect, each commissure bar includes a distal portion and a proximal portion, wherein the prosthetic valve is operably coupled to the distal portion, and wherein the proximal portion in the radially compressed configuration includes a first strut and a second strut separated by a slot.

In a fourth aspect, in the transcatheter heart valve prosthesis according to the third aspect, in the radially expanded configuration, the first strut and the second strut extend circumferentially in opposite directions from the distal portion of the commissure bar.

In a fifth aspect, in the transcatheter valve prosthesis according to any of the first through fourth aspects, in the radially expanded configuration, the outflow portion is spaced the third distance from the central longitudinal axis.

In a sixth aspect, in the transcatheter valve prosthesis according to any of the first through fifth aspects, the plurality of commissure bars comprises three commissure bars.

In a seventh aspect, in the transcatheter valve prosthesis according to any one of the first through sixth aspects, the prosthetic valve comprises a plurality of leaflets.

In an eighth aspect, in the transcatheter valve prosthesis according to the fifth aspect, commissures of the plurality of leaflets are coupled to the commissure bars of the frame.

In a ninth aspect, in the transcatheter valve prosthesis according to any one of the first through eighth aspects, the third distance is greater than the first distance.

In a tenth aspect, the transcatheter heart valve prosthesis according to any of the preceding aspects further includes a transition portion coupling the inflow portion to the outflow portion, wherein in the radially compressed configuration the transition portion is spaced the first distance from the central longitudinal axis, and in the radially expanded configuration, the transition portion tapers from the second distance from the central longitudinal axis to the third distance from the longitudinal axis.

In an eleventh aspect, the transcatheter heart valve prosthesis according to any of the preceding aspects further includes connectors coupling the commissure bars to the inflow portion.

In a twelfth aspect, in the transcatheter heart valve prosthesis according to the eleventh aspect, the connectors include an undulation region.

In a thirteenth aspect, in the transcatheter heart valve prosthesis according to any of the preceding aspects, each commissure bar includes holes and/or longitudinal struts.

In a fourteenth aspect, the transcatheter heart valve prosthesis according to any of the preceding aspects further includes cleats extending radially outwardly and proximally from the inflow portion.

In a fifteenth aspect, a transcatheter heart valve prosthesis includes a frame having a central longitudinal axis. The frame includes a plurality of inner axial struts, a plurality of outer axial struts, and a plurality of connectors coupling the inner axial struts to the outer axial struts. A prosthetic valve is operatively connected to at least some of the inner axial struts. In a radially compressed configuration, the plurality of inner axial struts, the plurality of outer axial struts, and the connectors are spaced a first distance from the central longitudinal axis. In a radially expanded configuration, the plurality of outer axial struts are spaced a second distance from the central longitudinal axis greater than the first distance, and the plurality of inner axial struts are spaced a third distance from the central longitudinal axis smaller than the second distance.

In a sixteenth aspect, in the transcatheter heart valve prosthesis according to the fifteenth aspect, the frame includes an inflow portion coupled to the plurality outer axial struts, wherein in the radially compressed configuration, the inflow portion is spaced the first distance from the central longitudinal axis, and wherein in the radially expanded configuration, the inflow portion is spaced the second distance from the central longitudinal axis.

In a seventeenth aspect, in the transcatheter heart valve prosthesis according to the fifteenth aspect or the sixteenth aspect, the frame includes an outflow portion coupled to the plurality inner axial struts, wherein in the radially compressed configuration, the outflow portion is spaced the first distance from the central longitudinal axis, and wherein in the radially expanded configuration, the outflow portion is spaced the third distance from the central longitudinal axis.

In an eighteenth aspect, in the transcatheter heart valve prosthesis according to the sixteenth aspect or the seventeenth aspect, the inflow portion includes a plurality of generally diamond shaped cells.

In a nineteenth aspect, a transcatheter heart valve prosthesis includes a frame having a central longitudinal axis and a prosthetic valve coupled to the frame. In a radially expanded configuration, the frame includes an outer structure and an inner structure, wherein the prosthetic valve is attached to the inner structure, and wherein the outer structure surrounds the inner structure and is spaced from the inner structure by a gap. In a radially compressed configuration, the outer structure and the inner structure are spaced about the same distance from the central longitudinal axis such that the gap disappears.

In a twentieth aspect, in the transcatheter heart valve prosthesis according the nineteenth aspect, the frame in the radially compressed configuration is a single layer tube.

In a twenty-first aspect, a transcatheter heart valve prosthesis includes a frame having a central longitudinal axis and a prosthetic valve coupled to the frame. In a radially compressed configuration, the frame is a single layer tube, and in a radially expanded configuration, the frame incudes an outer structure and an inner structure. The prosthetic valve is attached to the inner structure, and the outer structure surrounds the inner structure and is spaced from the inner structure by a gap.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the art to make and use the invention. The drawings are not to scale.

FIG. 1 depicts a perspective view of an embodiment of a transcatheter heart valve prosthesis in an expanded configuration in accordance with embodiments hereof.

FIG. 2 depicts a perspective view of a frame of the transcatheter heart valve prosthesis of FIG. 1 in the radially expanded configuration in accordance with embodiments hereof.

FIG. 3 depicts a perspective view of the frame of the transcatheter heart valve prosthesis of FIG. 1 in a radially compressed configuration in accordance with embodiments hereof.

FIG. 4 depicts a side view of the frame of the transcatheter heart valve prosthesis of FIG. 1 in the radially expanded configuration in accordance with embodiments hereof.

FIG. 5 depicts a top view of the frame of the transcatheter heart valve prosthesis of FIG. 1 in the radially expanded configuration in accordance with embodiments hereof.

FIG. 6 depicts a top view of the frame of the transcatheter heart valve prosthesis of FIG. 1 in the radially compressed configuration in accordance with embodiments hereof.

FIG. 7 depicts the frame of the transcatheter heart valve prosthesis of FIG. 1 in a laid open, flat view depicting a laser-cutting pattern in accordance with embodiments hereof.

FIG. 8 depicts a perspective view of a transcatheter heart valve prosthesis in a radially expanded configuration in accordance with embodiments hereof.

FIG. 9 depicts a perspective view of the frame of the transcatheter heart valve prosthesis of FIG. 8 in the radially expanded configuration in accordance with embodiments hereof.

FIG. 10 depicts a perspective view of the frame of the transcatheter heart valve prosthesis of FIG. 8, in a radially compressed configuration in accordance with embodiments hereof.

FIG. 11 depicts a side view of the frame of the transcatheter heart valve prosthesis of FIG. 8 in the radially expanded configuration in accordance with embodiments hereof.

FIG. 12 depicts a top view of the frame of the transcatheter heart valve prosthesis of FIG. 8 in the radially expanded configuration in accordance with embodiments hereof.

FIG. 13 depicts a top view of the frame of the transcatheter heart valve prosthesis of FIG. 8 in the radially compressed configuration in accordance with embodiments hereof.

FIG. 14 depicts the frame of the transcatheter heart valve prosthesis of FIG. 8 in a laid open, flat view depicting a laser-cutting pattern in accordance with embodiments hereof.

FIG. 15 depicts a perspective view of a transcatheter heart valve prosthesis in a radially expanded configuration in accordance with embodiments hereof.

FIG. 16 depicts a perspective view of a frame of the transcatheter heart valve prosthesis of FIG. 15 in the radially expanded configuration in accordance with embodiments hereof.

FIG. 17 depicts a side view of the frame of the transcatheter heart valve prosthesis of FIG. 15 in the radially expanded configuration in accordance with embodiments hereof.

FIG. 18 depicts a top view of the frame of the transcatheter heart valve prosthesis of FIG. 15 in the radially expanded configuration in accordance with embodiments hereof.

FIG. 19 depicts a perspective view of the frame of the transcatheter heart valve prosthesis of FIG. 15 in a radially compressed configuration in accordance with embodiments hereof.

FIG. 20 depicts a portion of the frame of the transcatheter heart valve prosthesis of FIG. 15 in a laid open, flat view depicting a laser-cutting pattern in accordance with embodiments hereof.

### DETAILED DESCRIPTION

Specific embodiments of the present invention are now described with reference to the figures, wherein like reference numbers indicate identical or functionally similar elements. The terms “inflow” and “outflow”, when used in the following description to refer to a valve prosthesis are with reference to the direction of blood flow. Thus, “inflow” refers to positions in an upstream direction with respect to the blood flow and “outflow” refers to positions in a downstream direction with respect to blood flow. Similarly “proximal” and “distal”, when used in the following description to refer to a valve prosthesis are with reference to the direction of blood flow. Thus, “proximal” refers to positions in an upstream direction with respect to the blood flow and “distal” refers to positions in a downstream direction with respect to blood flow.

The following detailed description is merely exemplary in nature and is not intended to limit the invention or the application and uses of the invention. Although the description of the invention is in the context of treatment of a native mitral heart valve, the invention may also be used where it is deemed useful in other valves that are not in the heart. For example, the present invention may be applied to other heart valves or venous valves as well. Furthermore, there is no intention to be bound by any expressed or implied theory presented in the preceding technical field, background, brief summary, or the following detailed description.

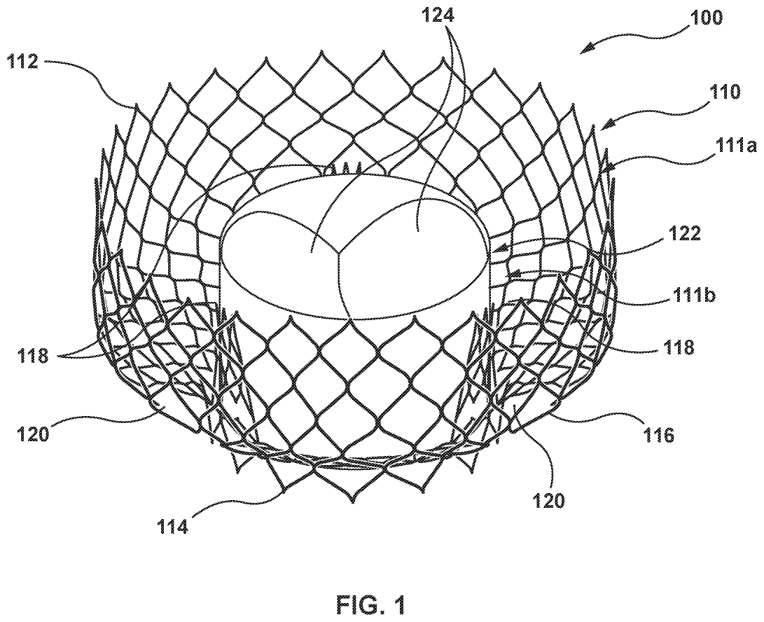

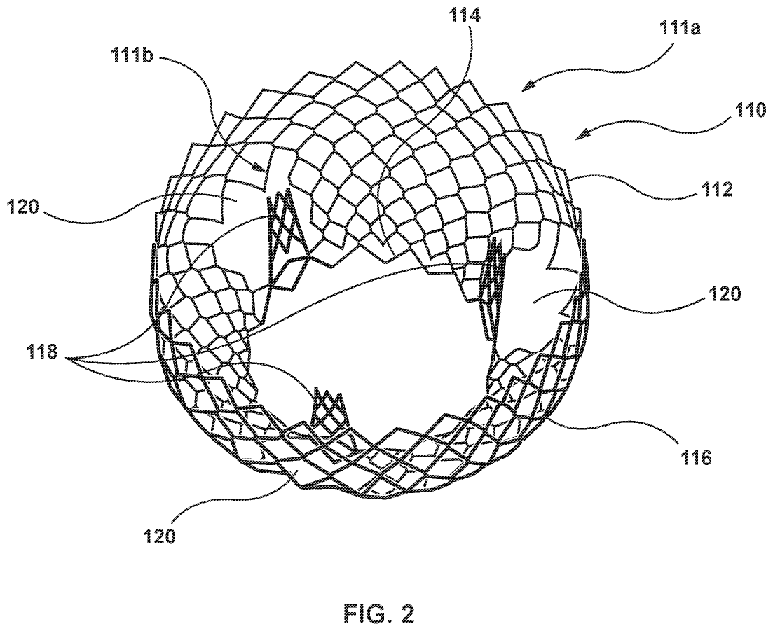

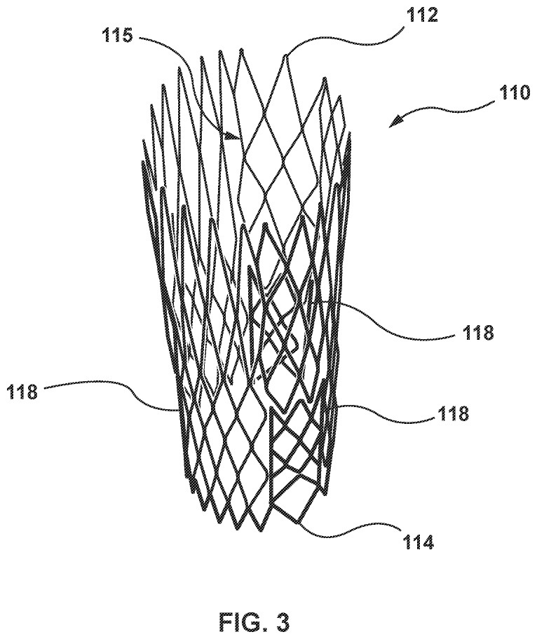

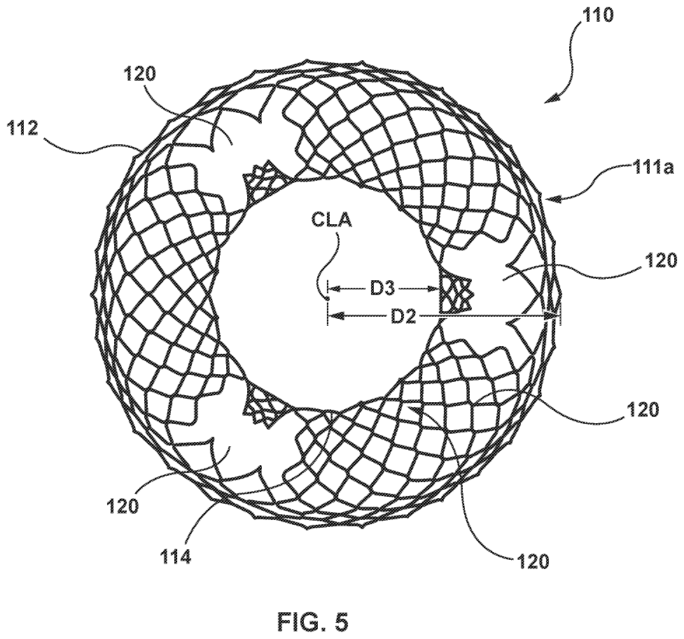

In an embodiment shown in FIGS. 1-7, a heart valve prosthesis 100 includes a radially expandable frame 110 and a prosthetic valve 122 coupled to the frame 110. The expandable frame 110 has a radially expanded configuration as shown in FIG. 2, for deployment within the native heart valve, and a radially compressed configuration, shown in FIG. 3, for delivery within the vasculature. Once delivered to the desired native valve, the frame 110 is deployed from the catheter and radially expands within the native valve leaflets of the native valve as to permanently place the native valve in an open state. The frame includes a central lumen 115.

FIGS. 2, 4, and 5 show the frame 110 in the radially expanded configuration. The frame in the radially expanded configuration includes an outer structure 111a and an inner structure 111b. As explained in more detail below, the outer and inner structures 111a, 111b are formed from a singular or unitary frame or tube.

The outer structure 111a, when the frame 110 is in the expanded configuration, is configured to be positioned against the walls of the native heart and to hold the inner structure 111b in a relatively consistent position within the heart. To be more specific, the outer structure 111a is relatively flexible, allowing for the outer structure 111a to absorb the natural movement of the heart as the heart contracts and relaxes. The inner structure 111b is generally positioned within outer structure 111a and is configured to hold the prosthetic valve 122 and remain relatively still as to allow the prosthetic valve 122 to replicate the function of the native valve.

The frame 110 includes an inflow portion 112, an outflow portion 114, and a transition portion 116. The inflow portion 112 is generally cylindrical, defines an inflow end of the frame 110, and is spaced a second distance D2 from the central longitudinal axis CLA in the radially expanded configuration. The outflow portion 114 is generally cylindrical, defines an outflow end of the frame 110, and is spaced a third distance D3 from the central longitudinal axis CLA in the radially expanded configuration, with the third distance D3 being smaller than the second distance D2. The transition portion 116 connects the inflow portion 112 and the outflow portion 114, and has an inflow end at an outflow end of the inflow portion that is spaced the second distance D2 from the central longitudinal axis CLA, and an outflow end at the inflow end of the outflow portion that is spaced the third distance D3 from the central longitudinal axis CLA. Thus, in the radially expanded configuration, the transition portion 116 tapers from the second distance D2 from the central longitudinal axis CLA to the third distance D3 from the central longitudinal axis CLA. If the inflow portion 112 and outflow portion 114 are generally cylindrical, the second distance D2 and third distance D3 are a radius of the inflow portion 112 and the outflow portion 1146, respectively. Instead of referring to the second distance D2 and the third distance D3 from the central longitudinal axis CLA, the inflow portion 112 can be referred to as having a second diameter, the outflow portion 114 having a third diameter smaller than the first diameter, and the transition portion 116 tapering from the second diameter to the third diameter. The outer structure 111a also includes openings 120. The openings 120 accommodate the inner structure 111b when the frame 110 is in the radially compressed configuration, as explained below.

In the embodiment of FIGS. 1-7, the inflow portion 112 includes a plurality of rows of generally diamond-shaped cells 113 formed from a plurality of rows of struts and crowns. In the embodiment shown, the inflow portion 112 includes at least three rows of cells 113, but this is not mean to be limiting. Similarly, the outflow portion 114 of the frame 110 includes a single row of struts and crowns. However, this is not meant to be limiting, and the outflow portion may include more rows of struts and crowns. Further, although shown as rows of struts and crowns forming cells, the outer structure 111a can include other stent structures known to those skilled in the art.

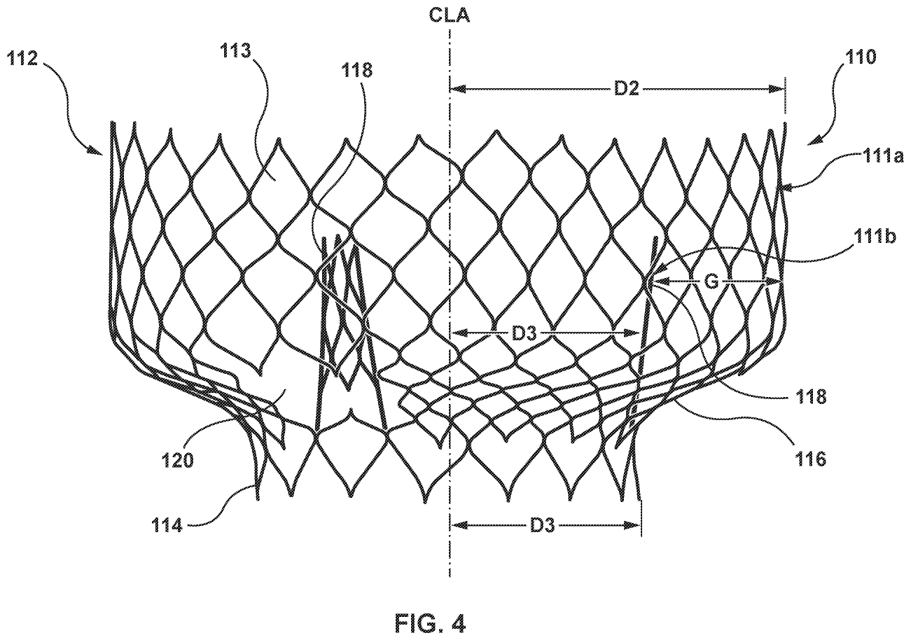

In the embodiment of FIGS. 1-7, the frame 110 in the radially expanded configuration includes the inner structure 111b, as shown in FIGS. 2, 4, and 5, the inner structure 111b includes three commissure bars 118 and the outflow portion 114. The commissure bars 118 extend proximally (i.e., in an upstream direction or towards the inflow end) from the outflow portion 114 of the frame 110. The prosthetic valve 122 is attached to the commissure bars 118. In particular, the commissures of the leaflets 124 of the prosthetic valve 122 are attached to a respective commissure bar 118 to secure the prosthetic valve 122 to the frame 110. As shown in FIG. 4, each commissure bar 118 is positioned the third distance D3 from the central longitudinal axis CLA. However as can be seen in FIG. 4, the commissure bars 118 in the radially expanded configuration may flare slightly outwardly in the upstream direction such that an upstream end of each commissure bar 118 is spaced a slightly greater distance from the central longitudinal axis CLA than the third distance D3. Nevertheless, even with such a flare, the upstream ends of the commissure bars 118 are spaced a distance from the central longitudinal axis CLA that is smaller than the second distance D2 such that the commissure bars 118 are positioned or spaced radially inwardly of the inflow portion 112 of the outer structure 111a. In other words, in the radially expanded configuration, a gap G is defined radially between the commissure bars 118 and the inflow portion 112 of the outer structure 111a, as shown in FIG. 4. The prosthetic valve 122 may be secured to the commissure bars 118 using sutures or stiches. In the embodiment shown, because the outflow portion 114 is spaced the third distance D3 from the central longitudinal axis, the outflow portion 114 can be described as part of the inner structure 111b. However, in other embodiments, the outflow portion 314 may be described as part of the outer structure 111a.

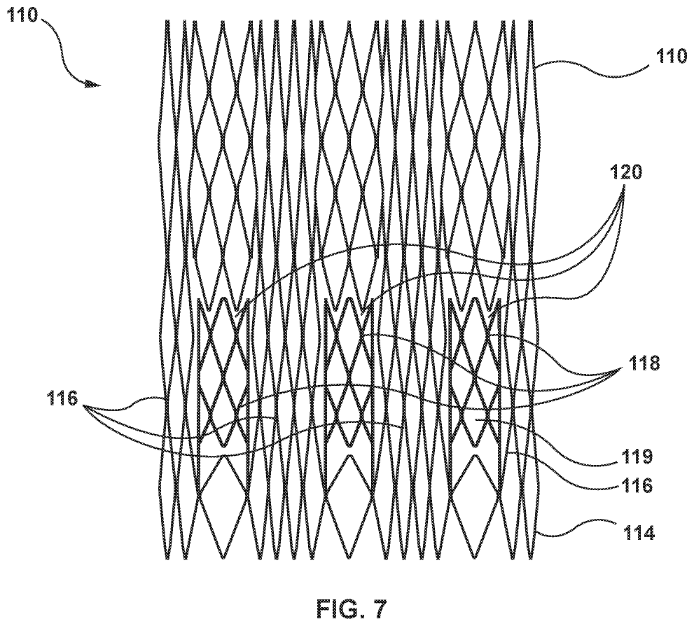

In the embodiment of FIGS. 1-7, the commissure bars 118 are formed from struts and crowns forming cells 119, as shown in FIG. 7. However, this is not mean to be limiting, and other structures may be used for the commissure bars 118, such as, but not limited to, axial struts.

Thus, as described above and shown in FIGS. 1, 3, 4, and 5, the frame 110 in the radially expanded configuration includes the outer structure 111a and the inner structure 111b, similar to a dual stent prosthesis described above. However, as explained in more detail below, the frame 110 is formed from a singular tube and in the radially compressed configuration, the inner and outer structures 111b, 111a are spaced about the same distance from the central longitudinal axis CLA such that in the radially compressed configuration, there is no “inner” structure and “outer” structure as the portions of the frame 110 that comprise the inner and outer structures 111b, 111a in the radially expanded configuration are spaced equally from the central longitudinal axis CLA in the radially compressed configuration. In other words, the frame 110 in the radially compressed configuration is a single-layer tube.

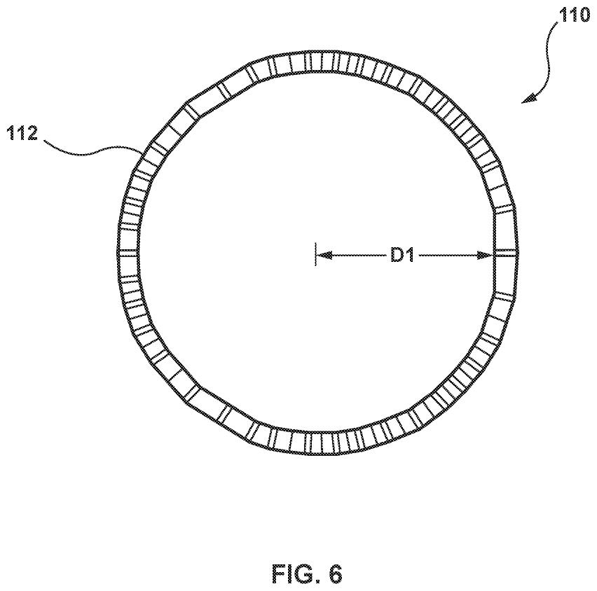

In particular, FIGS. 2 and 6 show the frame 110 of the heart valve prosthesis 100 in the radially compressed configuration. Further, FIG. 7 shows the frame 110 in a laid open, flat view. As best shown in FIGS. 2 and 7, in the radially compressed configuration, the commissures 118 are disposed within the openings 120. Thus, in the radially compressed configuration, the inflow portion 112, the transition portion 116, the outflow portion 114, and the commissure bars 118 are all spaced a first distance D1 from the central longitudinal axis CLA of the frame, as shown in FIG. 6. In other words, in the radially compressed configuration, the frame 110 is substantially cylindrical, with the inflow portion 112, the transition portion 116, the outflow portion 114, and the commissure bars 118 all having about the same diameter.

The frame 110 may be formed out of a singular or unitary tube. In particular, the frame 110 may be formed by laser-cutting, etching (such as chemical etching) or otherwise removing material from a tube in a pattern to form the frame 110 shown in FIG. 2. FIG. 7 shows the pattern of material removed from the tube with the tube in a laid open, flat view. Thus, as shown in FIG. 7 and FIG. 2, the material is removed from the tube in the inflow portion 112 of the frame 110 in a pattern to match the inflow portion 112 in the radially compressed configuration. Similarly, the material is removed from the tube in the outflow portion 114 in a pattern to match the outflow portion 114 of the frame 110. In the embodiment of FIGS. 1-7, the transition portion 116 and the commissures 118 are formed from the same longitudinal section of the tube. Thus, as shown in FIGS. 2 and 7, the transition portion 116 and the commissures 118 are circumferentially adjacent to each other around a circumference of the tube. Thus, the transition portion 116 and the commissures 118 are circumferentially adjacent to each other when the frame 110 is in the radially compressed configuration. As shown in FIGS. 2 and 7, around the circumference of the tube prior to removing material or around the circumference of the frame 110 after the material has been removed and the frame 110 is in the radially compressed configuration, there are three sections of the transition portion 116 and three sections of the commissures 118 alternatingly disposed around the circumference of the frame 110. As can be seen in FIG. 7, the commissures 118 are not circumferentially attached to the transition portion 116. Instead the commissures 118 are only attached to the outflow section 114, thereby forming the openings 120 in the frame 110 when the frame 110 radially expands, as explained above.

Having removed material from the tube in the pattern described above leaves the frame 110 in the radially compressed configuration, as shown in FIG. 2. For the frame 110 to radially expand to the radially expanded configuration shown in FIGS. 1, 3, 4, and 5, the frame 110 may be “shape-set” into the radially expanded configuration. For example, dies, mandrels, or other tools in the art may be used to manipulate and hold the frame 110 into the radially expanded configuration. Then, the frame 110 may be heat treated to shape-set the frame 110 into the radially expanded configuration, as known to those skilled in the art. Thus, for example, the frame 110 may be manipulated and held such that the inflow portion 112 is spaced the second distance D2 from the central longitudinal axis CLA, the outflow portion 114 is spaced the third distance D3 from the central longitudinal axis CLA, the commissures 118 are spaced the third distance D3 from the central longitudinal axis CLA and spaced from the inflow portion 112 by the gap G, and the transition portion 116 transitions from the inflow portion 112 to the outflow portion 114. Then, upon shape-setting, the frame 110 will return to the radially expanded configuration unless an outside force prevents the frame 110 from being in the radially expanded configuration. For example, and not by way of limitation, the frame 110 may be radially compressed and inserted into a catheter for delivery. Thus, the force of the catheter maintains the frame 110 in the radially compressed configuration. Once delivered to the treatment site, the portion of the catheter surrounding the frame 110 may be removed, such as by retraction, to release the frame 110 from the catheter such that the frame 110 will self-expand to the radially expanded configuration. The frame 110 may be made from self-expanding materials known in the art, such as but not limited to, stainless steel, nickel titanium alloys such as Nitinol™, or any number of other self-expanding biocompatible materials or combination of self-expanding biocompatible materials.

As explained above, the prosthetic valve 122 is positioned within the frame 110 and is coupled to the frame 110 at the commissure bars 118. The valve 122 includes one or more leaflets 124 that are attached to the frame 110 using sutures or other suitable attachment mechanisms. The leaflets 124 replicate the function of the native leaflets and are configured to open and close with the changing blood pressure in the heart when the heart valve prosthesis 100 is deployed at the treatment site. The leaflets 124 may be formed of various flexible materials including, but not limited to natural pericardial material such as tissue from bovine, equine or porcine origins, or synthetic materials such as polytetrafluorethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to stretching, deforming, or fatigue.

Those skilled in the art will recognize that FIGS. 1-8 illustrate one example of a transcatheter heart valve prosthesis and that existing components illustrated in FIGS. 1-8 may be removed and/or additional components may be added. For example, and not by way of limitation, the heart valve prosthesis 100 may further include a brim that may extend outwardly from the inflow portion 112. The brim may act as or assist maintain the position of the valve prosthesis 100 and may be configured to engage tissue above a native annulus, such as a supra-annular surface or other tissue in the left atrium. The brim thereby would inhibit the downstream migration of the heart valve prosthesis 100, for example, during atrial systole. Details and examples of brims may be found in U.S. Patent Application Publication No. 2016/0038280 A1, published Feb. 11, 2016, which is incorporated by reference herein in its entirety. In another example, and not by way of limitation, the heart valve prosthesis 100 may further include a skirt coupled to an inner surface or an outer surface of the frame 110. The skirt may be operatively connected to the inflow portion 112, the outflow portion 114, and/or the transition portion 116. The skirt acts as a seal around the heart valve prosthesis 100 to limit potential paravalvular leaks. The skirt may be a natural or biological material such as pericardium or another membranous tissue such as intestinal submucosa. Alternatively, the skirt may be a low-porosity woven fabric, such as polyester, Dacron fabric or PTFE, which creates a one-way fluid passage when attached to the stent. In one embodiment, the skirt may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side.

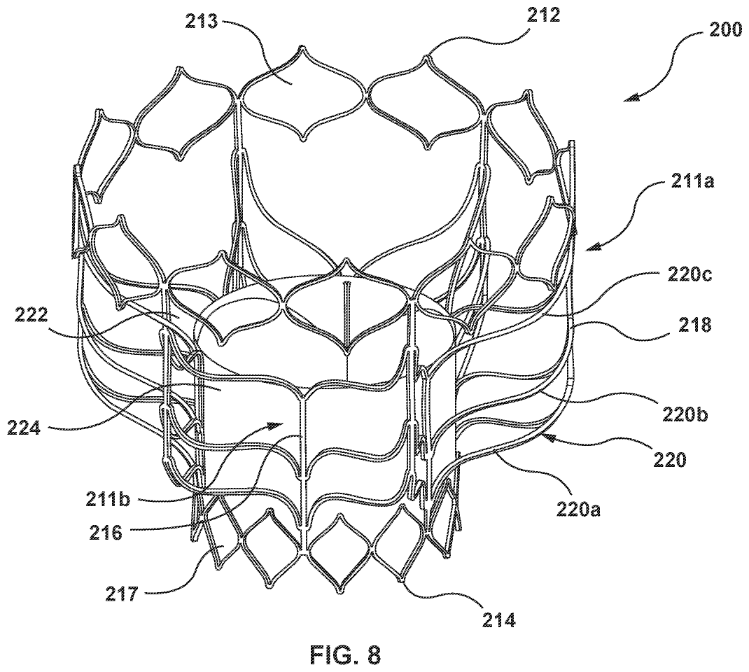

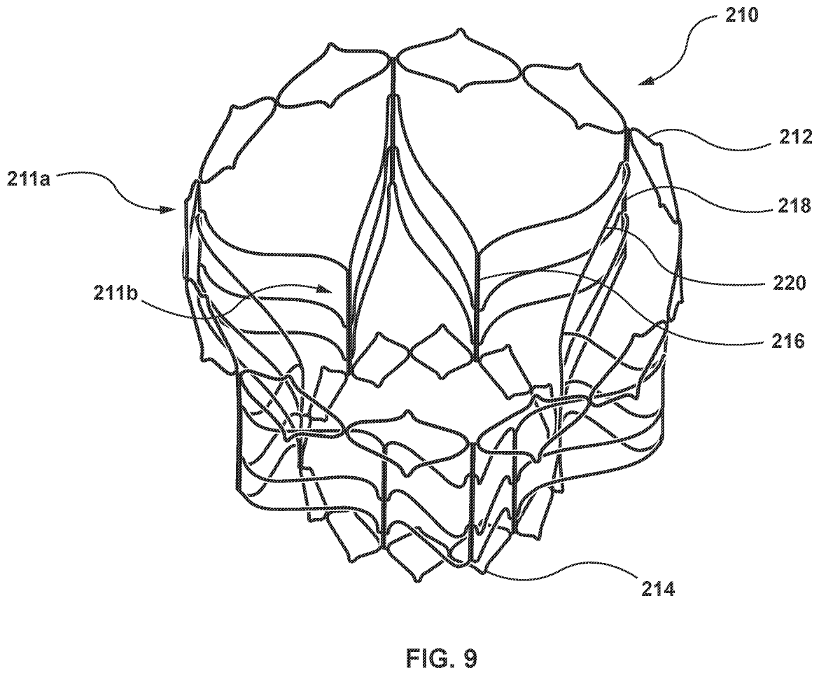

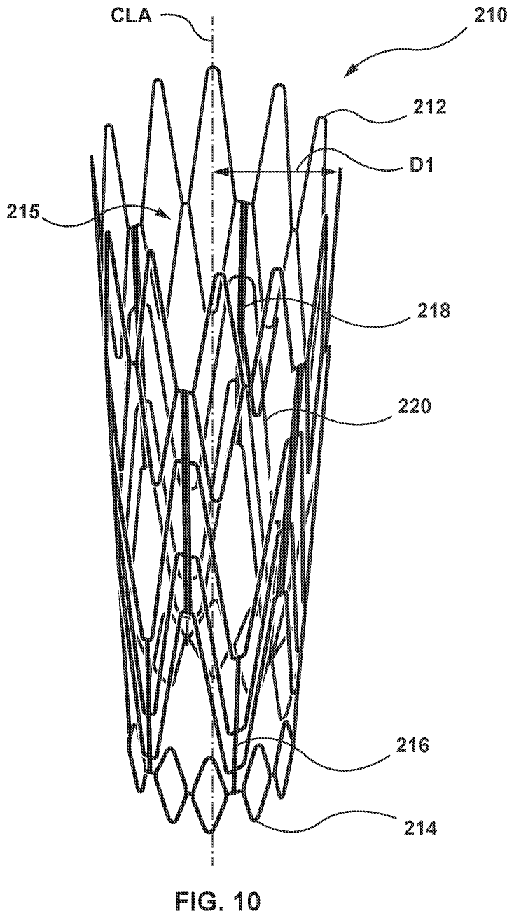

FIGS. 8-14 show another embodiment of a heart valve prosthesis 200. The heart valve prosthesis 200 includes a frame 210 and a prosthetic valve 222 coupled to the frame 210. The frame 210 (and thus the heart valve prosthesis 200) has a radially expanded configuration, shown in FIG. 9, for deployment within the native heart valve, and a radially compressed configuration, shown in FIG. 10, for delivery within the vasculature. Once delivered to the desired native valve, the frame 210 is deployed from the catheter and radially expands within the native heart valve leaflets of the native heart valve to place the native heart valve in an open state. The prosthetic valve 222 replaces or supplements the function of the native heart valve leaflets. The frame includes a central lumen 215.

FIGS. 8, 9, 11, and 12 show the frame 210 of the heart valve prosthesis 200 in a radially expanded configuration. In the radially expanded configuration, the frame 210 includes an outer structure 211a, an inner structure 211b, and connectors 220 that couple the outer structure 211a and the inner structure 211b.

The outer structure 211a, when the frame 210 is in the expanded configuration, is configured to be positioned against the walls of the native heart and to hold the inner structure 211b in a relatively consistent position within the heart. To be more specific, the outer structure 211a is relatively flexible, allowing for the outer structure 211a to absorb the natural movement of the heart as the heart contracts and relaxes. The inner structure 211b is generally positioned within outer structure 211a and is configured to hold the prosthetic valve 222 and remain relatively still as to allow the prosthetic valve 222 to replicate the function of the native valve.

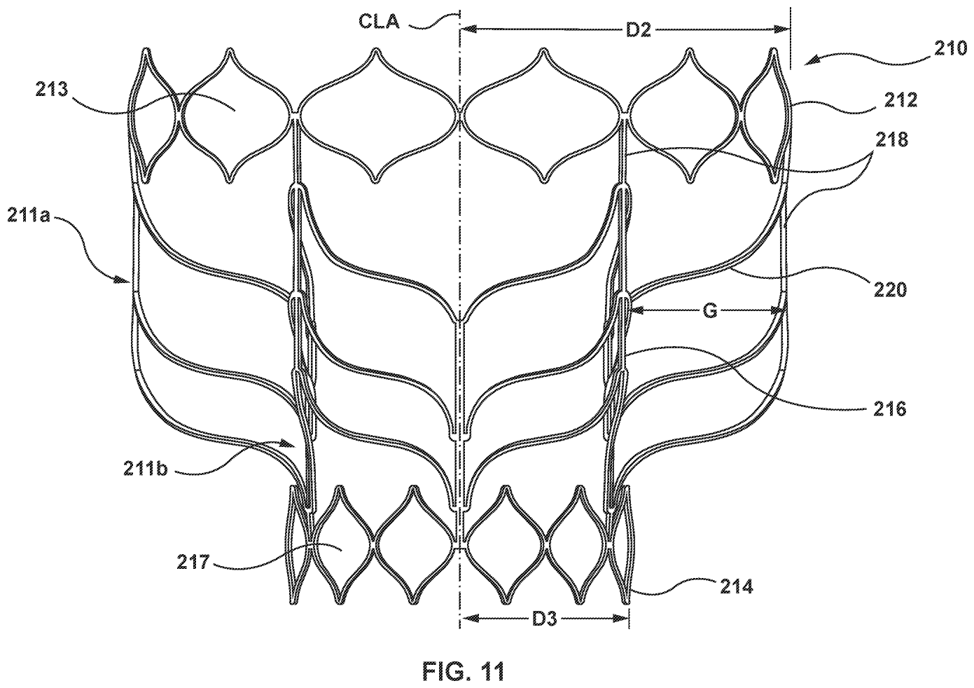

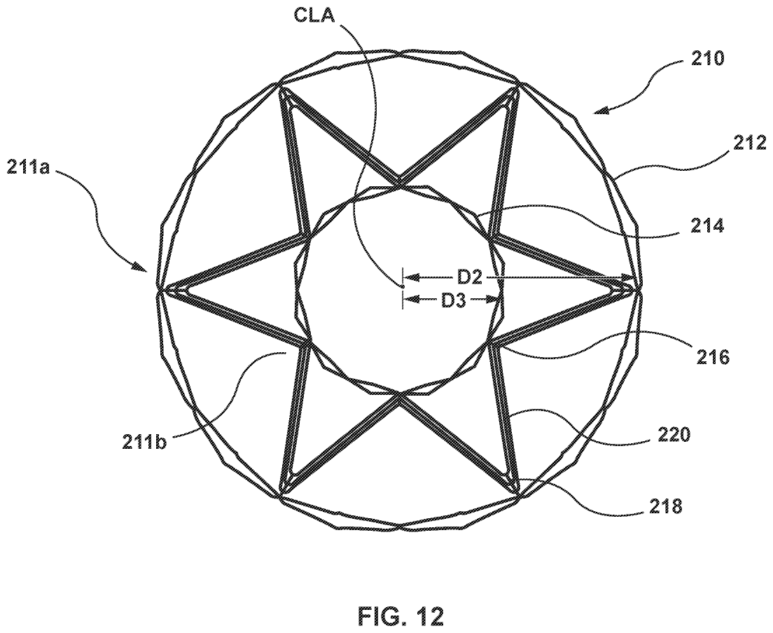

As shown in FIGS. 8, 9, 11, and 12, when in the radially expanded configuration, the outer structure 211a includes an inflow portion 212 a plurality of outer axial struts 218 coupled to the inflow portion 212 and extending distally therefrom. In the embodiment shown in FIGS. 8-14, the inflow portion 212 defines an inflow end of the frame 210 and is a single row of generally diamond-shaped cells 213 formed by two rows of struts and crowns. However, this is not meant to be limiting, and the inflow portion 212 may include more rows of cells, shapes other than generally diamond-shaped, and/or more rows of struts and crowns. The outer axial struts 218 are substantially parallel to the central longitudinal axis CLA. In the embodiment of FIGS. 8-14, the outer axial struts 218 extend distally from a joint between circumferentially adjacent cells 213 of the inflow portion 212. However, this is not meant to be limiting, and the outer axial struts 218 may be coupled to other portions of the inflow portion 212, such as, but not limited to, crowns thereof. As shown in FIGS. 11 and 12, when the frame is in the radially expanded configuration, the inflow portion 212 and the outer axial struts 218 are spaced a second distance D2 from the central longitudinal axis CLA of the frame 210.

When the frame 210 is in the radially expanded configuration, as shown in FIGS. 8, 9, 11, and 12, the frame 210 includes the inner structure 211b. The inner structure 211b includes an outflow portion 214 and a plurality of inner axial struts 216 extending proximally from the outflow portion 214. In the embodiment shown in FIGS. 8-14, the outflow portion 214 defines an outflow end of the frame 210 and is a single row of generally diamond-shaped cells 217 formed by two rows of struts and crowns. However, this is not meant to be limiting, and the outflow portion 212 may include more rows of cells, shapes other than generally diamond-shaped, and/or more rows of struts and crowns. The inner axial struts 216 are substantially parallel to the central longitudinal axis CLA. In the embodiment of FIGS. 8-14, the inner axial struts 216 extend proximally from a joint between circumferentially adjacent cells 217 of the outflow portion 214. However, this is not meant to be limiting, and the inner axial struts 216 may be coupled to other portions of the outflow portion 214, such as, but not limited to, crowns thereof. The prosthetic valve 222 is attached to at least some of the inner axial struts 216, as explained in more detail below. As shown in FIGS. 11 and 12, when the frame 210 is in the radially expanded configuration, the inner axial struts 216 and the outflow portion 214 are spaced a third distance D3 from the central longitudinal axis CLA. The third distance D3 is smaller than the second distance D2. In other words, the inner axial struts 216 and the outflow portion 214 are spaced radially inwards of the outer axial struts 218 and the inflow portion 212. As shown in FIG. 11, the inner axial struts 216 are spaced from the outer axial struts 218 by a gap G.

Further, in the embodiment of FIGS. 8-14, the inner axial struts 216 and the outer axial struts 218 are positioned in an alternating pattern around the circumference of the frame 210, as explained and shown in the radially compressed configuration. In the embodiment of FIGS. 8-14, the frame 210 has six inner axial struts 216 and six outer axial struts 218. However, this is not meant to be limiting, and the frame 210 may include more or fewer inner axial struts 216 and outer axial struts 218.

As shown in FIGS. 8, 9, 11 and 12, a plurality of connectors 220 are coupled to the inner axial struts 216 and the outer axial struts 218, thereby coupling the outer structure 211a to the inner structure 211b. In the embodiment of FIGS. 8-14, each connector 220 includes a first end attached to one of the inner axial struts 216 and a second end attached to one of the outer axial strut 218. The connectors 220 are arranged as chevrons such that the location of the second end of the one of the connectors 220 attached to an outer axial strut 218, a circumferentially adjacent connector 220 is attached and extends to a circumferentially adjacent inner axial strut 216. Thus, as can be seen in FIG. 8, circumferentially adjacent connectors 220 form a V-shape. Further, in the embodiment of FIGS. 8-14, there are three connectors 220 longitudinally between each one of the inner axial struts 216 and a circumferentially adjacent outer axial strut 218. Thus, as can be seen in FIG. 8, there is a first connecter 220a adjacent the outflow portion 214, a second connector 220b spaced distally from the first connector 220a, and a third connector spaced distally from the second connector 220b, with each attached to the same inner axial strut 216 and the same outer axial strut 218. However, this is not meant to be limiting, and more or fewer connectors 220 may be used connect each inner axial strut 216 to a corresponding one of the outer axial struts 218.

Thus, as described above and shown in FIGS. 8, 9, 11, and 12, the frame 210 in the radially expanded configuration includes the outer structure 211a and the inner structure 211b spaced inwardly from the outer structure 211A, similar to a dual stent prosthesis described above. However, as explained in more detail below, the frame 210 is formed from a singular tube and in the radially compressed configuration, the inner and outer structures 211b, 211a and the connectors 220 are spaced about the same distance from the central longitudinal axis CLA such that in the radially compressed configuration, there is no “inner” structure and “outer” structure as the portions of the frame 210 that comprise the inner and outer structures 211b, 211a in the radially expanded configuration are spaced equally from the central longitudinal axis CLA when in the radially compressed configuration.

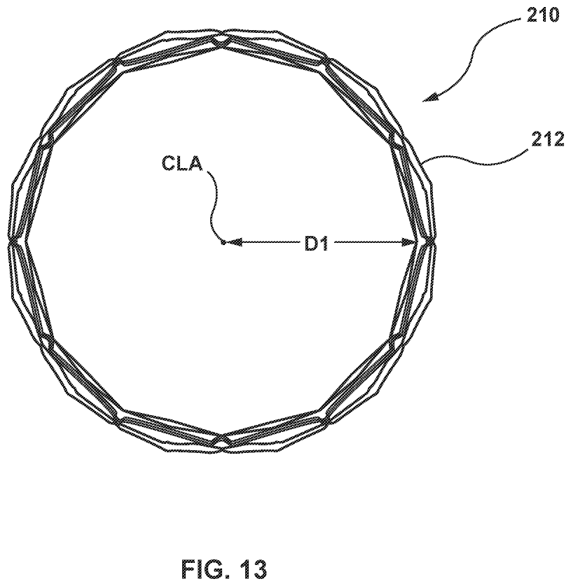

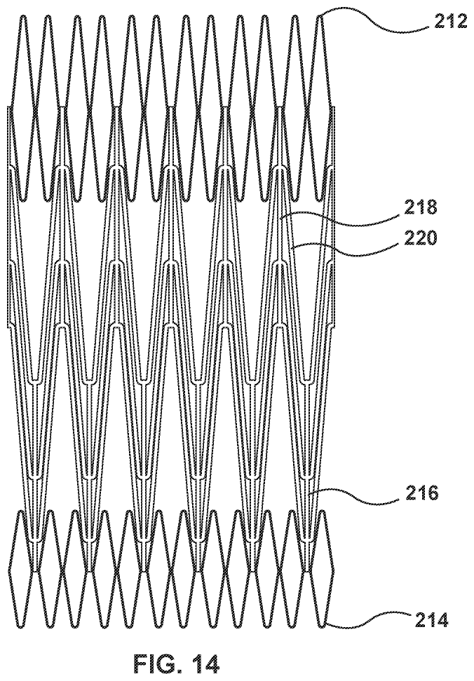

In particular, FIGS. 10 and 13 show the frame 210 of the heart valve prosthesis 200 in the radially compressed configuration. Further, FIG. 14 shows the frame 210 in a laid open, flat view. As best shown in FIGS. 10 and 14, in the radially compressed configuration, the inflow portion 212, the outer radial struts 218, the outflow portion 214, the inner axial struts 216, and the connectors 220 are all spaced a first distance DI from the central longitudinal axis CLA of the frame 210, as shown in FIGS. 10 and 13. The first distance D1 is smaller than the second distance D2. In other words, in the radially compressed configuration, the frame 210 is substantially cylindrical, with the inflow portion 212, the outflow portion 214, the outer axial struts 218, the inner axial struts 216, and the connectors 220 all having about the same diameter, with that diameter being smaller than the diameter of the inflow portion 212 and the outer axial struts 218 in the radially expanded configuration.

The frame 210 may be formed out of a singular or unitary tube. In particular, the frame 210 may be formed by laser-cutting, etching (such as chemical etching) or otherwise removing material from a tube in a pattern to form the frame 210 shown in FIG. 10. FIG. 14 shows the pattern of material removed from the tube with the tube in a laid open, flat view. Thus, as shown in FIG. 14 and FIG. 10, the material is removed from the tube in the inflow portion 212 of the frame 210 in a pattern to match the inflow portion 212 in the radially compressed configuration. Similarly, the material is removed from the tube in the outflow portion 214 in a pattern to match the outflow portion 214 of the frame 210 in the radially compressed configuration. In the embodiment of FIGS. 8-14, material is removed from the tube between the inflow portion 212 and the outflow portion 212 in a pattern to match the outer axial struts 218, the inner axial struts 216, and the connectors 220 in the radially compressed configuration. Further, in the embodiment shown, the outer axial struts 218 are formed partially in the inflow portion 212 and the inner axial struts 216 are formed partially in the outflow portion 214, as shown in FIG. 14, due to the attachment location of the outer and inner axial struts 218, 216 to the inflow and outflow portions 212, 214, respectively. As explained above, and shown in FIG. 14, the inner and outer axial struts 216, 218 are alternatingly disposed around the circumference of the frame 210.

Having removed material from the tube in the pattern described above leaves the frame 210 in the radially compressed configuration, as shown in FIG. 10. For the frame 210 to radially expand to the radially expanded configuration shown in FIGS. 8, 9, 11, and 12, the frame 210 may be “shape-set” into the radially expanded configuration. For example, dies, mandrels, or other tools in the art may be used to manipulate and hold the frame 210 into the radially expanded configuration. Then, the frame 210 may be heat treated to shape-set the frame 210 into the radially expanded configuration, as known to those skilled in the art. Thus, for example, the frame 210 may be manipulated and held such that the inflow portion 212 and the outer axials struts 218 are spaced the second distance D2 from the central longitudinal axis CLA, the outflow portion 114 and the inner axial struts 216 are spaced the third distance D3 from the central longitudinal axis CLA, and the connectors 220 are moved such that they span the gap G between the outer axial struts 218 and the inner axial struts 216. Then, upon shape-setting, the frame 210 will return to the radially expanded configuration unless an outside force prevents the frame 210 from being in the radially expanded configuration. For example, and not by way of limitation, the frame 210 may be radially compressed and inserted into a catheter for delivery. Thus, the force of the catheter maintains the frame 210 in the radially compressed configuration. Once delivered to the treatment site, the portion of the catheter surrounding the frame 210 may be removed, such as by retraction, to release the frame 210 from the catheter such that the frame 210 will self-expand to the radially expanded configuration. The frame 210 may be made from self-expanding materials known in the art, such as but not limited to, stainless steel, nickel titanium alloys such as Nitinol™, or any number of other self-expanding biocompatible materials or combination of self-expanding biocompatible materials.

As explained above, the prosthetic valve 222 is positioned within the frame 210 and is coupled to the frame 210. In the embodiment of FIGS. 8-14, the prosthetic valve 222 is coupled to at least some of the inner axial struts 216. The valve 222 includes one or more leaflets 224 that are attached to the frame 210 using sutures or other suitable attachment mechanisms. In particular, in the embodiment shown, the prosthetic valve includes three leaflets 224. Each commissure of the leaflets 224 is attached at one of the inner axial struts 216. Therefore, in the embodiment shown with three leaflets 224, the three commissures thereof are attached at a respective one of the inner axial struts 216. In the embodiment shown, there are six inner axial struts 216 and the commissures of the leaflets 224 are attached to every other one of the inner axial struts 216. Thus, around the circumference of the inner structure 211b in the radially expanded configuration, the inner axial struts 216 alternate between an inner axial strut 216 with a commissure of the leaflets 224 attached thereto and an inner axial strut 216 without a commissure of the leaflets 224 attached thereto. The embodiment shown and described is not meant to be limiting, and more or fewer leaflets and/or more or fewer inner axial struts may be utilized. The leaflets 224 replicate the function of the native leaflets and are configured to open and close with the changing blood pressure in the heart when the heart valve prosthesis 200 is deployed at the treatment site. The leaflets 224 may be formed of various flexible materials including, but not limited to natural pericardial material such as tissue from bovine, equine or porcine origins, or synthetic materials such as polytetrafluorethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to stretching, deforming, or fatigue.

Those skilled in the art will recognize that FIGS. 8-14 illustrate one example of a transcatheter heart valve prosthesis and that existing components illustrated in FIGS. 8-14 may be removed and/or additional components may be added. For example, and not by way of limitation, the heart valve prosthesis 200 may further include a brim that may extend outwardly from the inflow portion 212. The brim may act as or assist maintain the position of the valve prosthesis 100 and may be configured to engage tissue above a native annulus, such as a supra-annular surface or other tissue in the left atrium. The brim thereby would inhibit the downstream migration of the heart valve prosthesis 200, for example, during atrial systole. Details and examples of brims may be found in U.S. Patent Application Publication No. 2016/0038280 A1, published Feb. 11, 2016, which is incorporated by reference herein in its entirety. In another example, and not by way of limitation, the heart valve prosthesis 200 may further include a skirt coupled to an inner surface or an outer surface of the frame 210. The skirt may be operatively connected to the inflow portion 212, the outflow portion 214, the inner axial struts 216, and/or the outer axial struts 216. The skirt acts as a seal around the heart valve prosthesis 200 to limit potential paravalvular leaks. The skirt may be a natural or biological material such as pericardium or another membranous tissue such as intestinal submucosa. Alternatively, the skirt may be a low-porosity woven fabric, such as polyester, Dacron fabric or PTFE, which creates a one-way fluid passage when attached to the stent. In one embodiment, the skirt may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side.

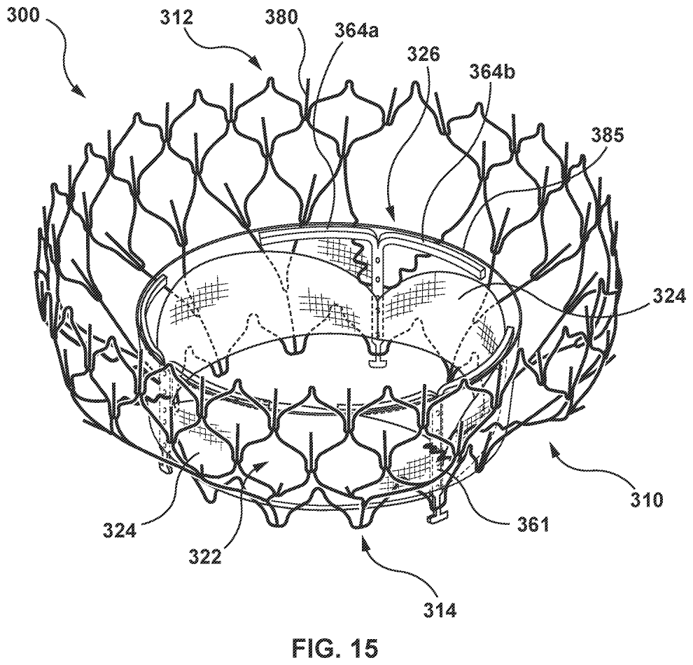

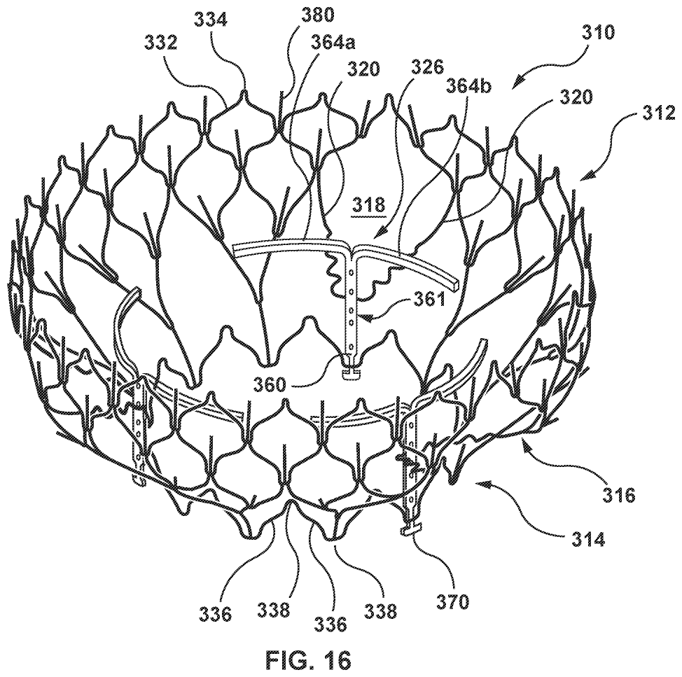

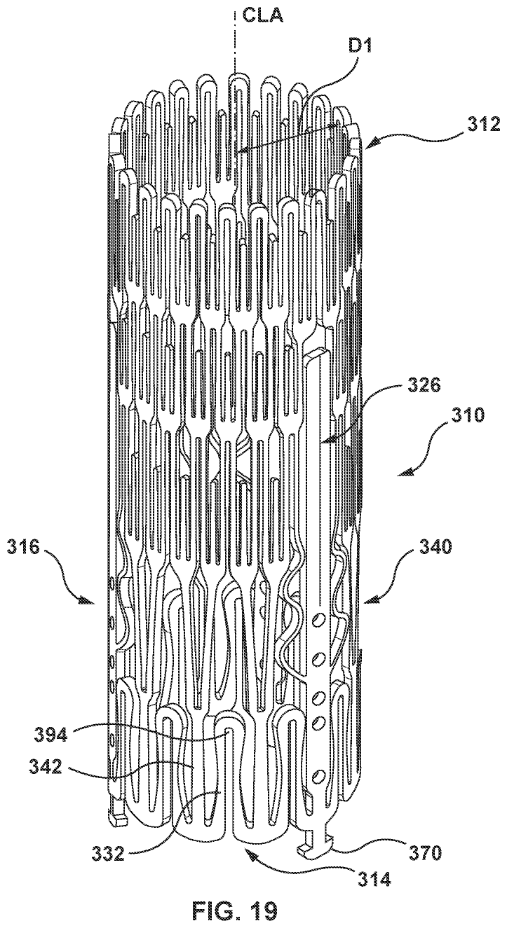

FIGS. 15-20 show another embodiment of a heart valve prosthesis 300. The heart valve prosthesis 300 includes a frame 310 and a prosthetic valve 322 coupled to the frame 310. The frame 310 (and thus the heart valve prosthesis 300) has a radially expanded configuration, shown in FIG. 16, for deployment within the native heart valve, and a radially compressed or radially unexpanded configuration, shown in FIG. 19, for delivery within the vasculature. Once delivered to the desired native heart valve, the frame 310 is deployed from the catheter and radially expands within the native heart valve leaflets of the native heart valve to place the native heart valve in an open state. The prosthetic valve 322 replaces or supplements the function of the native heart valve leaflets. The frame 310 further defines a central lumen 315 extending therethrough.

FIGS. 15-18 show the frame 310 of the heart valve prosthesis 300 in a radially expanded configuration. In the radially expanded configuration, the frame 310 includes an outer structure 311a, an inner structure 311b, and connectors 320 that couple the outer structure 311a and the inner structure 311b.

The outer structure 311a, when the frame 310 is in the expanded configuration, is configured to be positioned against the walls of the native heart and to hold the inner structure 311b in a relatively consistent position within the heart. To be more specific, the outer structure 311a is relatively flexible, allowing for the outer structure 311a to absorb the natural movement of the heart as the heart contracts and relaxes. The inner structure 311b is generally positioned within outer structure 311a and is configured to hold the prosthetic valve 322 and remain relatively still as to allow the prosthetic valve 322 to replicate the function of the native valve.

As shown in FIGS. 15-18, when in the radially expanded configuration, the outer structure 311a includes an inflow portion 312, an outflow portion 314, and a transition portion 316. In the embodiment shown, the inflow portion 312 is generally cylindrical, defines an inflow end of the frame 310, and is spaced a second distance D2 from the central longitudinal axis CLA in the radially expanded configuration. The outflow portion 314 is generally cylindrical, defines an outflow end of the frame 310, and is spaced a third distance D3 from the central longitudinal axis CLA in the radially expanded configuration, with the third distance D3 being smaller than the second distance D2. The transition portion 316 connects the inflow portion 312 and the outflow portion 314, and has an inflow end at an outflow end of the inflow portion 312 that is spaced the second distance D2 from the central longitudinal axis CLA, and an outflow end at the inflow end of the outflow portion 314 that is spaced the third distance D3 from the central longitudinal axis CLA. Thus, in the radially expanded configuration, the transition portion 316 tapers from the second distance D2 from the central longitudinal axis CLA to the third distance D3 from the central longitudinal axis CLA. If the inflow portion 312 and outflow portion 314 are generally cylindrical, the second distance D2 and third distance D3 are each a radius of the inflow portion 312 and the outflow portion 314, respectively. Instead of referring to the second distance D2 and the third distance D3 from the central longitudinal axis CLA, the inflow portion 312 can be referred to as having a second diameter, the outflow portion 314 having a third diameter smaller than the second diameter, and the transition portion 316 tapering from the second diameter to the third diameter. The outer structure 311a also includes openings 318. The openings 318 accommodate the inner structure 311b when the frame 310 is in the radially compressed configuration, as explained below.

In the embodiment of FIGS. 15-20, the inflow portion 312 includes a plurality of rows of struts 332 and bends 334, the bends 334 joining adjacent struts 332. In the embodiment shown, the rows of struts 332 and bends 334 form generally diamond-shaped cells 313. In the embodiment shown, the inflow portion 312 includes two rows of cells 313, but this is not meant to be limiting. Further, as can be seen in FIGS. 15-17, the rows of cells 313 are not complete rows of cells 313, as they are interrupted by the openings 318, explained in more detail below. Similarly, the outflow portion 314 of the frame 310 includes a single row of struts 336 and bends 338. However, this is not meant to be limiting, and the outflow portion 314 may include more rows of struts and bends. Further, although shown as rows of struts and bends forming cells, the outer structure 311a can include other stent structures known to those skilled in the art.

In the embodiment of FIGS. 15-20, the transition portion 316 includes a single row 340 of cells 313 and a plurality of axial struts 342 connecting the row 340 to the outflow portion 314. However, this is not meant to be limiting, as there may be additional or fewer rows 340 of cells 313 in the transition portion 316. Further, the row 340 is not a complete circumferential row as it is interrupted by the openings 318, described in more detail below. Further, the axial struts 342 are generally parallel to the central longitudinal axis CLA of the frame when in the radially compressed configuration, but are disposed at an angle in the radially expanded configuration to transition from the larger diameter inflow portion 312 to the smaller diameter outflow portion 314.

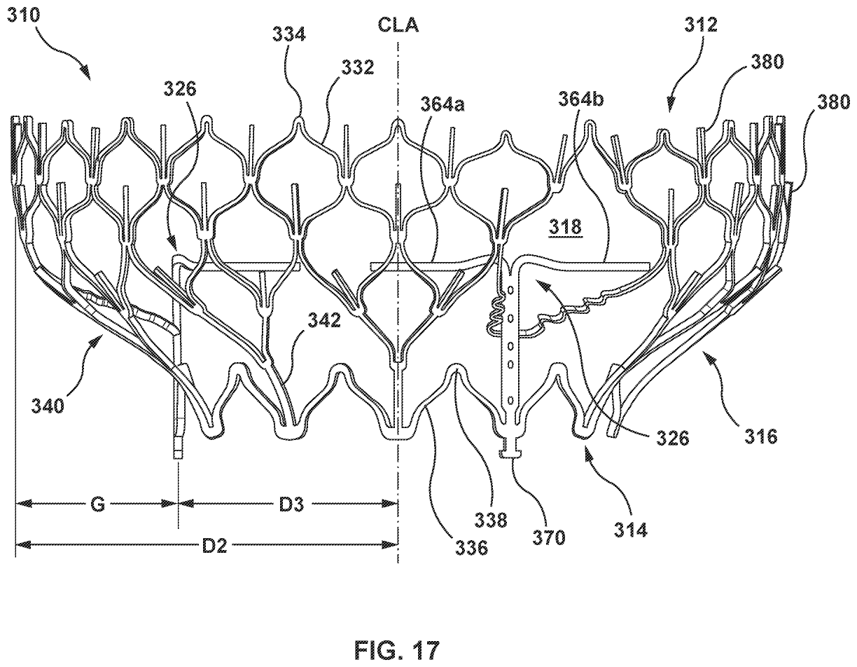

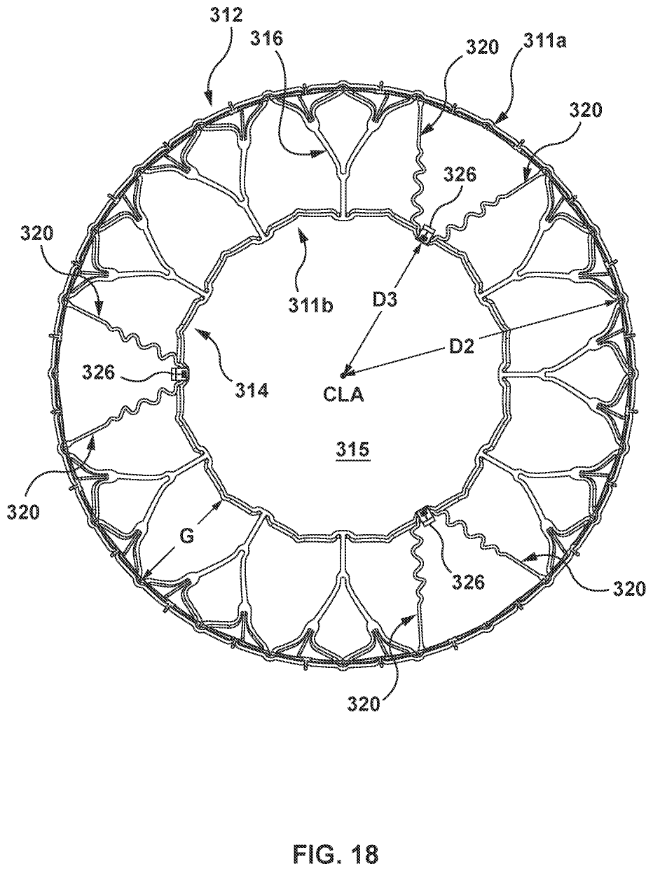

In the embodiment of FIGS. 15-20, the frame 310 in the radially expanded configuration includes the inner structure 311b, as shown in FIGS. 15-18. The inner structure 311b includes three commissure bars 326. The commissure bars 326 extend proximally (i.e., in an upstream direction or towards the inflow end) from the outflow portion 314 of the frame 110. The prosthetic valve 322 is attached to the commissure bars 326. In particular, the commissures of the leaflets 324 of the prosthetic valve 322 are attached to a respective commissure bar 326 to secure the prosthetic valve 322 to the frame 310. As shown in FIG. 18, each commissure bar 326 is positioned the third distance D3 from the central longitudinal axis CLA with the frame 310 in the radially expanded configuration. However as can be seen in FIG. 17, the commissure bars 326 with the frame 310 in the radially expanded configuration may taper slightly inwardly in the upstream direction such that an upstream end of each commissure bar 326 is spaced a slightly smaller distance from the central longitudinal axis CLA than the third distance D3. Nevertheless, even with such a taper, both the upstream and downstream ends of the commissure bars 326 are spaced a distance from the central longitudinal axis CLA that is smaller than the second distance D2 such that the commissure bars 326 are positioned or spaced radially inwardly of the inflow portion 312 of the outer structure 311a. In other words, in the radially expanded configuration, a gap G is defined radially between the commissure bars 326 and the inflow portion 312 of the outer structure 311a, as shown in FIGS. 17 and 18. The prosthetic valve 322 may be secured to the commissure bars 326 using sutures or stiches. In the embodiment shown, because the outflow portion 314 is spaced the third distance D3 from the central longitudinal axis, the outflow portion 314 can be described as part of the inner structure 311b. However, in other embodiments, the outflow portion 314 may be described as part of the outer structure 311a.

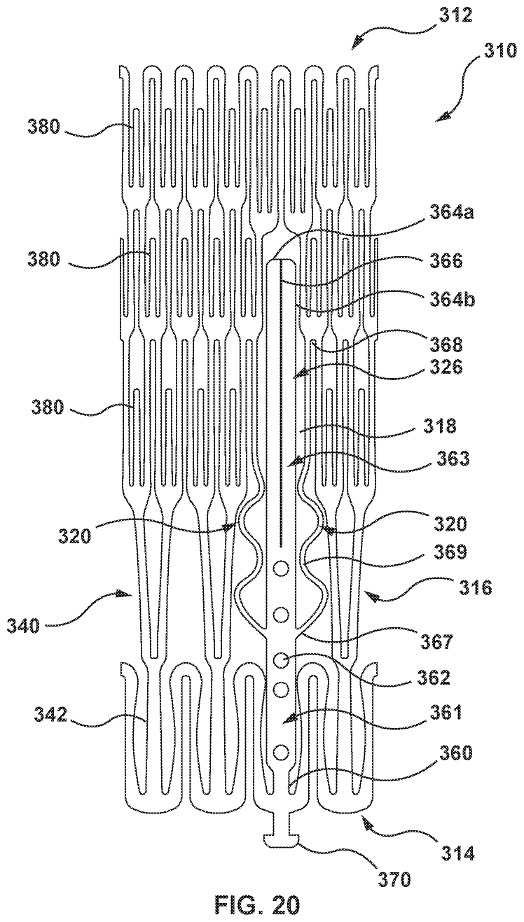

In the embodiment of FIGS. 15-20, the commissure bars 326 are axial struts or bars that form a commissure bar and circumferential supports when radially expanded. As can be seen best in FIG. 20, each commissure bar 326 includes a downstream or distal end 360 coupled to a bend 328 in the outflow portion 314. In particular, the distal end 360 of each commissure bar 326 is coupled to a valley of the row of struts and bends forming the outflow portion 314, as described above. However, this is not meant to be limiting, and the distal end 360 may be coupled to a peak of the outflow portion 314 or other portion of the frame 310. Further, each commissure bar 326 includes a distal portion 361 and a proximal portion 363. The distal portion 361 of each commissure bar 326 includes holes or openings 362 therethrough. The holes 362 may be utilized to attach the prosthetic valve 322 to the commissure bars 326 and/or to attach a skirt (not shown) to the commissure bars 326, and/or for other purposes. However, this is not meant to be limiting, and other ways to attach the prosthetic valve 322 to the distal portions 361 of the commissure bars 326 may be utilized, such as longitudinal slots, or simply attaching the prosthetic valve 322 to the distal portion 361, such as through stitching. The proximal portion 363 of each commissure bar 326 may include a longitudinal slot 366 therethrough, dividing the commissure bar into a first strut 364a and a second strut 364b. The first and second struts 364a, 364b are aligned substantially longitudinally in the radially compressed configuration, as shown in FIG. 19. However, upon expansion, the first and second struts 364a, 364b of each commissure bar 326 separate from each other, bend towards the outflow portion 314, and extend circumferentially, as shown in FIGS. 15 and 16. The proximal portion 363 with the first and second struts 364a, 364b provides additional structural support for the inner structure 311b when the frame 310 is in the radially expanded configuration. Further, the first and second struts 364a, 364b provide additional support for an inner skirt 385 attached to the inner structure 311b. Similar to the remainder of the frame 310, the first and second struts 364a, 364b are shape-set to the radially expanded configuration, shown in FIGS. 15-18, and are radially compressed to the radially compressed configuration, shown in FIG. 19.

As shown in FIGS. 15-20, a plurality of connectors 320 couple the commissure bars 326 to the inflow portion 312, thereby coupling the outer structure 311a to the inner structure 311b when the frame 310 is in the radially expanded configuration. In the embodiment of FIGS. 15-20, each connector 320 includes a first end 367 attached to the commissure bar 326 and a second end 368 attached to the inflow portion 314. More particularly, in the embodiment shown, the first end 367 is coupled to a distal portion of the commissure bar 326 and the second end 368 is coupled to the second row of cells 313 of the inflow portion 312. Thus, in the embodiment shown, the second end 368 is proximal to or upstream of the first end 367. However, this is not meant to be limiting, and the connectors 320 may be coupled to different portions of the commissure bar 326 and/or different portions of the outer structure 311a. In the embodiment shown, each connector further includes an undulating, wavy, or zig-zag region 369. The undulating region 369 assists in expansion and recapture of the transcatheter heart valve prosthesis 300. In the embodiment shown, there are two connectors 320 per commissure bar 326, extending from opposite lateral sides of the commissure bar. However, this is not meant to be limiting, and more connectors 320 may be provided for each commissure bar 326.

In the embodiment shown in FIGS. 15-20 the frame 310 further includes barbs or cleats 380 extending radially outward and proximally (upstream). In the embodiment of FIGS. 15-20, the cleats 380 extend from bends 334 of the second, third, and fourth rows of struts 32 and bends 334 of the inflow portion 312 of the frame 310. However, this is not meant to be limiting, and the cleats 380 may extend from different locations, or from fewer or more locations than shown. The cleats 380 are configured to engage tissue at the native heart valve to prevent longitudinal migration or displacement of the heart valve prosthesis 300 upon deployment at the native heart valve. For example, and not by way of limitation, the heart valve prosthesis 300 may be deployed within a native mitral valve. In such a situation, the cleats 380 engage the mitral annulus/upstream surface of the native mitral leaflets to prevent migration of the heart valve prosthesis 300 towards the left atrium. Similarly, if deployed at a native tricuspid valve, the cleats 380 are configured to prevent migration towards the right atrium. In other deployment locations, the cleats 380 may be direction downstream rather than upstream of the migration to be prevented is in the downstream direction.

In the embodiment of FIGS. 15-20, the frame 310 further includes tabs 370 extending distally from the commissure bars 326. The tabs 370 are configured to couple the frame 310 to a delivery catheter. For example, a spindle of a delivery catheter may include recesses with shapes corresponding to the shape of the tabs 370. During delivery of the heart valve prosthesis 300, the tabs 370 are disposed within such recesses. Upon expansion of the heart valve prosthesis 300, the tabs 370 expand out of the recesses. While three tabs 370 are shown corresponding to the three commissure bars 326, this is not meant to be limiting. Further, the tabs 370 need not be associated with the commissure bars 326. For example, and not by way of limitation, the frame may alternatively only include two tabs 370, with a first tab 370 extending distally from one of the commissure bars 326 and a second tab extending distally from a distalmost bend 338 of the outflow portion 314, 180 degrees apart from the first tab 370. Other arrangements are also contemplated. Further, instead of extending distally from an outflow end of the frame 310, the tabs 370 may instead extend proximally from a proximal end of the frame 310, such as from bends 334 of the inflow portion 312.

As described above and shown in FIGS. 15-18, the frame 310 in the radially expanded configuration includes the outer structure 311a and the inner structure 311b, similar to a dual stent prosthesis described above. However, as explained in more detail below, the frame 310 is formed from a singular tube and in the radially compressed configuration, the inner and outer structures 311b, 311a are spaced about the same distance from the central longitudinal axis CLA such that in the radially compressed configuration, there is no “inner” structure and “outer” structure as the portions of the frame 310 that comprise the inner and outer structures 311b, 311a in the radially expanded configuration are spaced approximately equally from the central longitudinal axis CLA in the radially compressed configuration. In other words, the frame 310 in the radially compressed configuration is a single-layer tube. This provides a smaller packing profile, thereby enabling a smaller diameter delivery catheter.

In particular, FIG. 19 shows the frame 310 of the heart valve prosthesis 300 in the radially compressed or radially unexpanded configuration. Further, FIG. 20 shows a portion of the frame 310 in a laid open, flat view. As best shown in FIGS. 19 and 20, in the radially compressed configuration, the commissures 326 are disposed within the openings 318. Thus, in the radially compressed configuration, the inflow portion 312, the transition portion 316, the outflow portion 314, and the commissure bars 326 are all spaced a first distance DI from the central longitudinal axis CLA of the frame, as shown in FIG. 19. In other words, in the radially compressed configuration, the frame 310 is substantially cylindrical, with the inflow portion 312, the transition portion 316, the outflow portion 314, and the commissure bars 326 all having about the same diameter. This single layer tube in the radially unexpanded configuration versus a “double layer” prosthesis in the radially expanded configuration is accomplished by the commissure bars 326 and connectors 320 being disposed in the openings 318 in the radially unexpanded configuration, and the frame 310 being shape set to expand to the radially expanded configuration with the different diameters noted above, as explained in more detail below.

In particular, the frame 310 may be formed out of a singular or unitary tube. For example, the frame 310 may be formed by laser-cutting, etching (such as chemical etching) or otherwise removing material from a tube in a pattern to form the frame 310 shown in FIG. 19. FIG. 20 shows a portion of the pattern of material removed from the tube with the tube in a laid open, flat view. Thus, as shown in FIG. 19 and FIG. 20, the material is removed from the tube in the inflow portion 312, the outflow portion 314, and the transition portion 316 of the frame 110 in a pattern to match the inflow portion 312, the outflow portion 314, and the transition portion 316 in the radially compressed configuration. Further, material is removed from the tube to form the openings 318, the connectors 320, and the commissure bars 326 in the radially compressed configuration.

Having removed material from the tube in the pattern described above leaves the frame 310 in the radially compressed configuration, as shown in FIG. 19. For the frame 310 to radially expand to the radially expanded configuration shown in FIGS. 15-18, the frame 310 may be “shape-set” into the radially expanded configuration. For example, dies, mandrels, or other tools in the art may be used to manipulate and hold the frame 310 into the radially expanded configuration. Then, the frame 310 may be heat treated to shape-set the frame 110 into the radially expanded configuration, as known to those skilled in the art. Thus, for example, the frame 310 may be manipulated and held such that the inflow portion 312 is spaced the second distance D2 from the central longitudinal axis CLA, the outflow portion 314 is spaced the third distance D3 from the central longitudinal axis CLA, the commissure bars 326 are spaced the third distance D3 from the central longitudinal axis CLA and spaced from the inflow portion 312 by the gap G, the transition portion 316 transitions from the inflow portion 312 to the outflow portion 314, and the connectors 320 are shape set to extend across the gap G from the inflow portion 312 to the commissure bars 326. Then, upon shape-setting to the radially expanded configuration, the frame 310 will return to the radially expanded configuration unless an outside force prevents the frame 310 from being in the radially expanded configuration. For example, and not by way of limitation, the frame 310 may be radially compressed and inserted into a catheter for delivery. Thus, the force of the catheter maintains the frame 310 in the radially compressed configuration. Once delivered to the treatment site, the portion of the catheter surrounding the frame 310 may be removed, such as by retraction, to release the frame 310 from the catheter. The frame 310 will self-expand to the radially expanded configuration. The frame 310 may be made from self-expanding materials known in the art, such as but not limited to, stainless steel, nickel titanium alloys such as Nitinol™, or any number of other self-expanding biocompatible materials or combination of self-expanding biocompatible materials.

As explained above, the prosthetic valve 322 is positioned within the frame 310 and is coupled to the frame 310 at the commissure bars 326. The valve 222 includes one or more leaflets 324 that are attached to the frame 310 using sutures or other suitable attachment mechanisms. The leaflets 324 replicate the function of the native leaflets and are configured to open and close with the changing blood pressure in the heart when the heart valve prosthesis 300 is deployed at the treatment site. The leaflets 324 may be formed of various flexible materials including, but not limited to natural pericardial material such as tissue from bovine, equine or porcine origins, or synthetic materials such as polytetrafluorethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to stretching, deforming, or fatigue.

Those skilled in the art will recognize that FIGS. 15-20 illustrate one example of a transcatheter heart valve prosthesis and that existing components illustrated in FIGS. 15-20 may be removed and/or additional components may be added. For example, and not by way of limitation, the heart valve prosthesis 300 may further include a brim that may extend outwardly from the inflow portion 312. The brim may act as or assist maintain the position of the valve prosthesis 300 and may be configured to engage tissue above a native annulus, such as a supra-annular surface or other tissue in the left atrium. The brim thereby would inhibit the downstream migration of the heart valve prosthesis 300, for example, during atrial systole. Details and examples of brims may be found in U.S. Patent Application Publication No. 2016/0038280 A1, published Feb. 11, 2016, which is incorporated by reference herein in its entirety. In another example, and not by way of limitation, the heart valve prosthesis 300 may further include a skirt coupled to an inner surface or an outer surface of the frame 310. The skirt may be operatively connected to the inflow portion 312, the outflow portion 314, and/or the transition portion 316. The skirt acts as a seal around the heart valve prosthesis 300 to limit potential paravalvular leaks. The skirt may be a natural or biological material such as pericardium or another membranous tissue such as intestinal submucosa. Alternatively, the skirt may be a low-porosity woven fabric, such as polyester, Dacron fabric or PTFE, which creates a one-way fluid passage when attached to the stent. In one embodiment, the skirt may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side.

As explained above, the frame of the heart valve prosthesis of each of the embodiments of FIGS. 1-8, 8-14, and 15-20 includes a radially expanded configuration having an inner structure and an outer structure disposed around and spaced from the inner structure. Having such inner and outer structures in the radially expanded configuration provides benefits of the dual stent prostheses described above. For example, and not by way of limitation, with the heart valve prosthesis in the radially expanded configuration deployed at the treatment site, the outer structures described above are positioned within the native annulus and/or within the native leaflets. The outer structures are relatively flexible such that they conform to the tissue at the native heart valve and absorb the natural movement of the heart while the inner structures can maintain their shape such that the prosthetic valve functions properly. However, in each of the embodiments of FIGS. 1-8, 8-14, and 15-20 the frame of the heart valve prosthesis in the radially compressed configuration is a single-layered tube. In other words, in the radially compressed configuration, the inner and outer structures of the frame are spaced approximately equally from the central longitudinal axis of the frame. Put another way, the inner and outer structures have the same diameter or transverse dimension in the radially compressed configuration. Such a single-layer tube in the radially compressed configuration reduces the total packing volume and the density of material of the heart valve prosthesis within a catheter relative to a dual-stent prosthesis. Smaller packing volumes lead to lower loading, deployment, and recapture forces. Further, smaller packing volumes enable the use of smaller diameter catheters for delivering the heart valve prosthesis. For example, and not by way of limitation, the heart valve prostheses disclosed herein may be delivered in 29 French catheters or 25 French catheters or less than 25 French catheters. For example, calculations for the transcatheter heart valve prosthesis 300 indicate that it can be delivered in a 15.1 French catheter. For comparison, traditional dual stent heart valve prostheses often need a 33 French catheter for delivery.

As used herein, the term “generally” and “substantially” mean approximately. When used to describe angles such as “substantially parallel” or “substantially perpendicular” the term “substantially” means within 10 degrees of the angle. When used to describe shapes such as “substantially” or “generally” cylindrical or “substantially” or “generally” tube-shaped, the terms mean that the shape would appear cylindrical to tube-shaped to a person of ordinary skill in the art viewing the shape with a naked eye. The term “about” as used herein to refer to dimensions means within 5% of the dimension.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Further, features described in one of the embodiments may be utilized in the other embodiments. For example, and not by way of limitation, the cleats described with respect to the heart valve prosthesis 300 may be added to the heart valve prosthesis 100 and/or the heart valve prosthesis 200. Thus, the breadth and scope of the present invention should not be limited by any one of the above-described exemplary embodiments, but should be understood that each feature of each embodiment discussed herein, and of each reference cited herein, can be used in combination with the features of any other embodiment. All patents and publications discussed herein are incorporated by reference herein in their entirety.

1. A transcatheter heart valve prosthesis comprising:
a frame having a central longitudinal axis, the frame including an inflow portion, an outflow portion, and a plurality of commissure bars extending from the outflow portion towards the inflow portion; and
a prosthetic valve operatively connected to the plurality of commissure bars,
wherein:
in a radially compressed configuration, each of the plurality of commissure bars is disposed within a respective opening of a plurality of openings in the frame, and the inflow portion, the outflow portion, and the plurality of commissure bars are spaced a first distance from a central longitudinal axis of the frame; and
in a radially expanded configuration, the inflow portion is spaced a second distance from the central longitudinal axis greater than the first distance, and the plurality of commissure bars are spaced a third distance from the central longitudinal axis of the frame less than the second distance.

2. The transcatheter heart valve prosthesis of claim 1, wherein the plurality of commissure bars extends substantially parallel to the central longitudinal axis.

3. The transcatheter heart valve prosthesis of claim 1, wherein each commissure bar includes a distal portion and a proximal portion, wherein the prosthetic valve is operably coupled to the distal portion, and wherein the proximal portion in the radially compressed configuration includes a first strut and a second strut separated by a slot.

4. The transcatheter heart valve prosthesis of claim 3, wherein in the radially expanded configuration, the first strut and the second strut extend circumferentially in opposite directions from the distal portion of the commissure bar.

5. The transcatheter heart valve prosthesis of claim 1, wherein in the radially expanded configuration, the outflow portion is spaced the third distance from the central longitudinal axis.

6. The transcatheter heart valve prosthesis of claim 1, wherein the plurality of commissure bars comprises three commissure bars.

7. The transcatheter heart valve prosthesis of claim 1, wherein the prosthetic valve comprises a plurality of leaflets.

8. The transcatheter heart valve prosthesis of claim 5, wherein respective commissures of the plurality of leaflets are coupled to respective commissure bars of the frame.

9. The transcatheter heart valve prosthesis of claim 5, wherein the third distance is greater than the first distance.

10. The transcatheter heart valve prosthesis of claim 5, further comprising a transition portion coupling the inflow portion to the outflow portion, wherein in the radially compressed configuration the transition portion is spaced the first distance from the central longitudinal axis, and in the radially expanded configuration, the transition portion tapers from the second distance from the central longitudinal axis to the third distance from the longitudinal axis.

11. The transcatheter heart valve prosthesis of claim 1, further comprising connectors coupling the commissure bars to the inflow portion.

12. The transcatheter heart valve prosthesis of claim 11, wherein the connectors include an undulation region.

13. The transcatheter heart valve prosthesis of claim 1, wherein each commissure bar includes holes and/or longitudinal slots.

14. The transcatheter heart valve prosthesis of claim 1, further comprising cleats extending radially outwardly and proximally from the inflow portion.

15. A transcatheter heart valve prosthesis comprising:
a frame having a central longitudinal axis, the frame a plurality of inner axial struts, a plurality of outer axial struts, and a plurality of connectors coupling the inner axial struts to the outer axial struts; and
a prosthetic valve operatively connected at least some of the inner axial struts;
wherein:
in a radially compressed configuration, the plurality of inner axial struts, the plurality of outer axial struts, and the connectors are spaced a first distance from the central longitudinal axis, and
in a radially expanded configuration, the plurality of outer axial struts are spaced a second distance from the central longitudinal axis greater than the first distance, and the plurality of inner axial struts are spaced a third distance from the central longitudinal axis smaller than the second distance.

16. The transcatheter heart valve prosthesis of claim 15, wherein the frame further comprises an inflow portion coupled to the plurality outer axial struts, wherein in the radially compressed configuration, the inflow portion is spaced the first distance from the central longitudinal axis, and wherein in the radially expanded configuration, the inflow portion is spaced the second distance from the central longitudinal axis.

17. The transcatheter heart valve prosthesis of claim 15, wherein the frame further comprises an outflow portion coupled to the plurality inner axial struts, wherein in the radially compressed configuration, the outflow portion is spaced the first distance from the central longitudinal axis, and wherein in the radially expanded configuration, the outflow portion is spaced the third distance from the central longitudinal axis.

18. The transcatheter heart valve prosthesis of claim 16, wherein the inflow portion comprises a plurality of generally diamond shaped cells.

19. A transcatheter heart valve prosthesis comprising:
a frame including a central longitudinal axis; and
a prosthetic valve coupled to the frame,
wherein:
in a radially expanded configuration, the frame comprises an outer structure and an inner structure, wherein the prosthetic valve is attached to the inner structure, and wherein the outer structure surrounds the inner structure and is spaced from the inner structure by a gap, and
in a radially compressed configuration, the outer structure and the inner structure are spaced about the same distance from the central longitudinal axis such that the gap disappears.

20. The transcatheter heart valve prosthesis of claim 19, wherein in the radially compressed configuration, the frame is a single layer tube.
